(19) United States
(12) Patent Application Publication
(10) Pub. No.: US 2026/0363563 A1
(43) Pub. Date: Nov. 19, 2026

(54) Systems and Methods for Mitigating Pressure Pulsations

(71) Applicant: Becton, Dickinson and Company, Franklin Lakes, NJ (US)

(72) Inventors: Huy Thanh Vu, Westminster, CA (US); Salvatore Manzella Jr., Aliso Viejo, CA (US); William Allen Rodemeyer, Corona, CA (US)

(21) Appl. No.: 19/478,177

(22) PCT Filed: Apr. 23, 2024
(86) PCT No.: PCT/US2024/025784

§ 371 Date: Oct. 23, 2025

Related U.S. Application Data
(60) Provisional application No. 63/497,919, filed on Apr. 24, 2023.

(51) Int. Cl. A61B 5/021; A61B 5/0235

(52) U.S. Cl. CPC A61B 5/02141; A61B 5/0235

(57) ABSTRACT

Systems and methods for damping pressure pulsations are provided. A damping system can comprise a system of expansion chambers with indirect ports within the chambers to reduce pressure pulsations. A release valve can be provided to reduce pressure pulsations as well.

### TECHNOLOGICAL FIELD

The current application claims the benefit of and priority to U.S. Provisional Patent Application No. 63/497,919 entitled “Systems and Methods for Mitigating Pressure Pulsations” filed Apr. 24, 2023, the disclosure of which is hereby incorporated by reference in its entirety for all purposes.

### TECHNOLOGICAL FIELD

The disclosure is generally directed to systems and methods for mitigating pressure pulsations, including mitigating pressure pulsations for use in blood pressure monitoring systems.

### BACKGROUND

Continuous noninvasive blood pressure monitors enable real-time measurement of blood pressure waves and derived hemodynamic parameters. Multiple techniques can be utilized including the volume clamp method.

The volume clamp method measures arterial blood pressure at an extremity (e.g., finger) utilizing an inflatable cuff, a light source (e.g., light emitting diode (LED)), and light sensor. The pressure in the cuff is adjusted to keep the diameter of the artery constant (the unloaded state), in which the diameter is determined using photoplethysmography via the light source and light sensor. The pressure within the inflatable cuff represents the arterial pressure of the finger artery. A pressure pump supplies the pressure to the inflatable cuff.

### SUMMARY

Systems and methods for damping pressure pulsations utilize a manifold comprising a set of expansion chambers and a set of unaligned ports between the expansion chambers. Systems and methods for damping pressure pulsations utilize a manifold and a release valve, the release valve configured to release excess pressure.

In some implementations, a damping system is for damping pressure pulsations from a pressure supply source. The damping system comprises a set of three or more expansion chambers are connected by a set of two or more ports between each of the expansion chambers. A first expansion chamber is configured to receive pressure and transfer that pressure to a second expansion chamber via a first port. The second expansion chamber is configured to transfer that pressure to a third expansion chamber via a second port. The first port and the second port are unaligned.

In some implementations, the second port is askew towards a side from the first port.

In some implementations, the second port is askew towards a top from the first port.

In some implementations, the second port is askew towards a bottom from the first port.

In some implementations, the first port or the second port contains a bend.

In some implementations, the set of three or more expansion chambers are provided within an internal compartment of a manifold.

In some implementations, the manifold contains an inlet port in fluidic connection with a pressure source.

In some implementations, the manifold contains an outlet port in fluidic connection with a pressure regulator.

In some implementations, the manifold contains an outlet port in fluidic connection with a medical device.

In some implementations, the medical device a pressure cuff for use within a blood pressure monitoring system.

In some implementations, a system is for reducing pressure pulsations from a pressure supply source. The system comprising a manifold in fluidic connection with a pressurized air source. The manifold is configured to passively dampen pressure pulsations via a set of one or more expansion chambers. The system further comprises a release valve provided after pressure source and either prior to or concurrently with an inlet to the manifold. The pressurized air source is configured to provide an excess volume of pressurized air that is greater than the volume capacity of the set of one or more expansion chambers. The release valve is configured to release the excess volume of pressurized air.

In some implementations, the release valve is configured to control the volume of pressurized air to be released.

In some implementations, the manifold contains an outlet port in fluidic connection with a pressure regulator.

In some implementations, the manifold contains an outlet port in fluidic connection with a medical device.

In some implementations, the medical device comprises a pressure cuff for use within a blood pressure monitoring system.

In some implementations, the pressured air source is configured to provide an excess volume of pressured air that is at least 1.0× greater than the volume capacity of the set of expansion chambers.

In some implementations, a method is for reducing pressure pulsations from a pressure supply source via the system for reducing pressure pulsations from a pressure supply source. The method comprising providing a volume of pressured air from the pressured air source. The volume of pressured air includes an excess volume such that the volume of pressured air provided is greater than the volume capacity of the set of one or more expansion chambers. The method further comprising releasing the excess volume of pressurized air via the release valve and transferring the remaining volume of pressurized air into the set of one or more expansion chambers.

In some implementations, the release valve releases at least 50% of the provided volume of pressured air.

In some implementations, the method further comprising further transferring the remaining volume of pressurized air into a pressure controller.

In some implementations, the method further comprising further transferring the remaining volume of pressurized air into a pressure cuff of a blood pressure monitoring system.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1 provides an example of pressure flow showing the pressure pulsations as generated by a pressure source.

FIG. 2 provides an exemplary blood pressure monitoring system incorporating a damping system.

FIGS. 3A, 3B, and 3C provide an example of a manifold for damping pressure pulsations via expansion chambers. FIG. 3A is a perspective view. FIG. 3B is a cross-sectional view. FIG. 3C is an exploded view.

FIGS. 4A to 4D provide examples of ports for use within a manifold for damping pressure pulsations via expansion chambers.

FIGS. 5A and 5B provide an example of a manifold for damping pressure utilizing a release valve. FIG. 5A is a perspective view. FIG. 5B is an exploded view.

### DETAILED DESCRIPTION

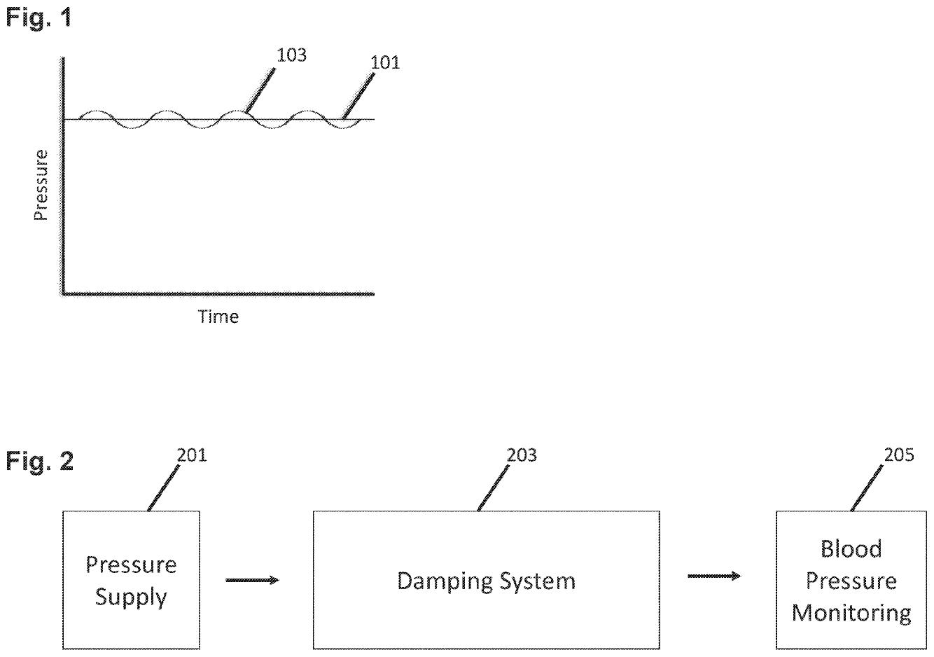

The current disclosure details systems and methods for mitigating pressure pulsations in a blood pressure monitoring system or other medical devices that utilize a pressure source. Pressure pumps and other pressure sources typically provide pulsating pressure, and although the pulsation is minor, it can cause inaccuracies in sensitive measurements and treatments. FIG. 1 provides an example of a graph with simulated data depicting continuous pressure measurement representative of a typical pressure source providing a constant pressure. While the average pressure 101 provided is the desired constant pressure out, that actual pressure output provided from the pump 103 is a sine wave, yielding a pressure pulsation effect of the average provided pressure. The pulsation can affect the sensitivity of medical devices, especially blood pressure monitoring systems.

It is a goal of the current application to reduce the pulsation effect that is provided by a pressure source via expansion chambers within a manifold, the chambers having particular port configurations that promote air to flow throughout the entire chamber. An expansion chamber system can comprise a plurality of chambers in which the ports between the chambers are indirect such that the air flow cannot take a direct path. The lack of direct path promotes air flow to expand throughout the chamber. The more the air flow expands, the more the chamber can passively dampen the pressure pulsation into a more smoothened pressure. The smoothened pressure will yield better accuracy when used in a blood pressure monitoring system or other medical device that benefits from less pressure pulsation.

It is also a goal to the current application to reduce the pulsation effect that is provided by a pressure source by having the pressure source generate an excess of volume that can be handled by the expansion chamber manifold. The larger amount of volume generated by a pressure (assuming constant amount of pressure) have relatively less pulsation. To reduce pressure pulsations, a volume of pressure can be generated by a pressure pump that is excessive of the expansion chamber manifold and a release valve can be provided after the pressure source and before an expansion manifold. The valve can release at least a portion of the excess volume such that the manifold intakes a volume of pressured air it can handle. The volume of pressure that is taken in by the manifold will have less pressure pulsation as compared to if the same volume was generated by a pressure pump and directly transferred to the manifold (assuming a constant amount of pressure).

The described systems, devices, and methods should not be construed as limiting in any way. Instead, the present disclosure is directed toward all novel and nonobvious features and aspects of the various disclosed systems and devices, either alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Various examples of systems and manifolds for decreasing pressure pulsation are disclosed herein, and any combination of these examples can be made unless specifically excluded. For example, any of the expansion chamber port configurations disclosed, can be used with a pressure release valve for releasing excess volume, even if a specific combination is not explicitly described. Likewise, the different constructions and features of manifolds for decreasing pressure pulsation can be mixed and matched, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

### Systems and Devices for Mitigating Pressure Pulsation

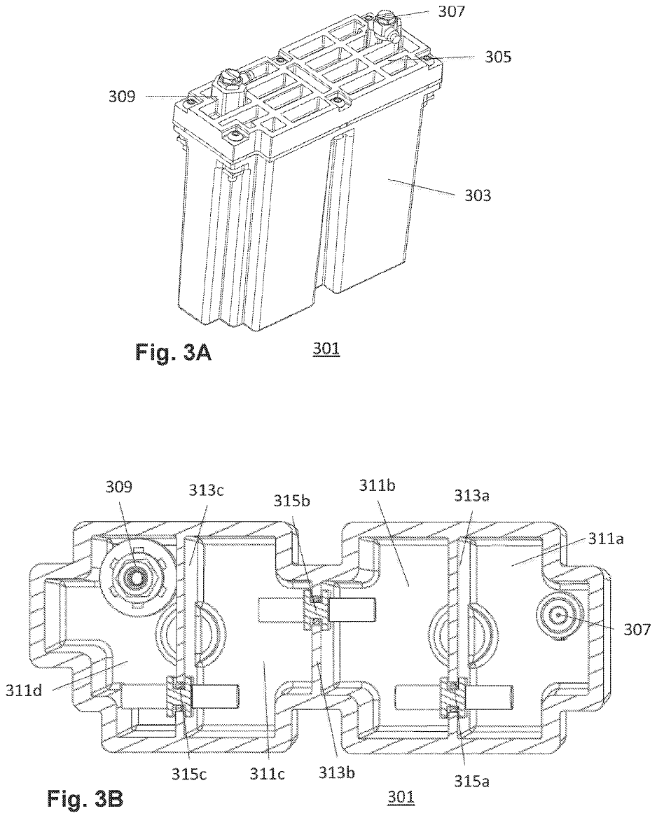

Provided in FIG. 2 is an overview system diagram of a pressure system utilized within a blood pressure monitoring system. The pressure system comprises a pressure supply 201 and a damping system 203. Pressure supply 201 can be any source that can provide pressure, such a pressure pump. The pressure pump can be a positive-displacement pump, a centrifugal pump, an axial-flow pump or any other pump capable of generating pressure. In some implementations, the pressure pump is a positive-displacement pump. Types of positive-displacement pumps that can be utilized include (but are not limited to) rotary-type pumps, reciprocating-type pumps, linear-type pumps, and pneumatic pumps. The various pumps each provide a relative amount of pressure pulsation as the pump mechanics facilitate the movement of fluid (liquid or gas).

The pressure provided by the pressure supply 201 is transferred through a damping system 203 to reduce the amount of pulsation of the pressure as provided by the supply. The damping system 203 can be provided as a set of expansion chambers within a manifold and/or a volume release valve. Generally, the set of expansion chambers comprises one or more chambers and when at least two chambers are utilized, the chambers are connected via ports. The ports can be configured in such a way the direction of flow is indirect between the chambers. The pressure release valve can be provided in between the pressure source and the expansion manifold.

When damping system 203 is utilized within blood pressure monitoring system, the pressure flow out of the active damping system is utilized for blood pressure monitoring 205. Generally, the damped pressure flow travels through a pressure regulator system that senses the pressure amount and can adjust the supplied pressure as necessary. The damped pressure is then transferred into a blood pressure cuff, which can be a cuff surrounding any extremity of a patient, such as (for example) an arm or a digit. In some instances, the blood pressure monitoring system utilizes a volume clamp method for continuous blood pressure monitoring and thus the pressure is adjusted based on the amount of pressure to keep the diameter of a patient's artery constant via the pressure cuff. When a volume clamp method is utilized, the supplied pressure to the cuff that keeps the artery diameter constant is the blood pressure within that artery.

In a typical continuous blood monitoring system utilizing the volume clamp method, the amount of pressure needed within the cuff will depend on the patient but will generally range between 60 mmHg (diastolic) and 120 mmHg (systolic) for healthy patients. Hypotensive patients may have diastolic pressure as low as 40 mmHg and hypertensive patients may have systolic pressure as high as 250 mmHg. Accordingly, the range of pressure to be provided to the cuff will between about 40 mmHg to about 250 mmHg. The pressure controller can receive an excess of pressure and then control the amount to be distributed to the pressure cuff. The amount of pressure generated by the pump can be higher such that the pressured air that travels through the damping system can lose a little bit of pressure along the circuit and still have the requisite excess pressure when it reaches the pressure system.

To provide the appropriate amount of pressure to the blood pressure cuff, the pump will need to provide enough volume of the pressured air, but in moments of extreme pressure changed, higher pressure may be needed. The slower the pump (i.e., less volume of pressure air generated), the greater the pressure pulsations. To ensure accurate pressure readings, it is ideal to keep the generated pressure at about the mid-range flow rate but can vary as dependent on the monitoring system. A passive damping system that comprises a manifold with a set of expansion chambers must be built to have an internal volume capacity to handle the volume of pressured air generated. In other words, the internal volume capacity within the expansion chambers needs to be able to handle the pressured air from the low to high flow rates, considering the amount of pressure supplied.

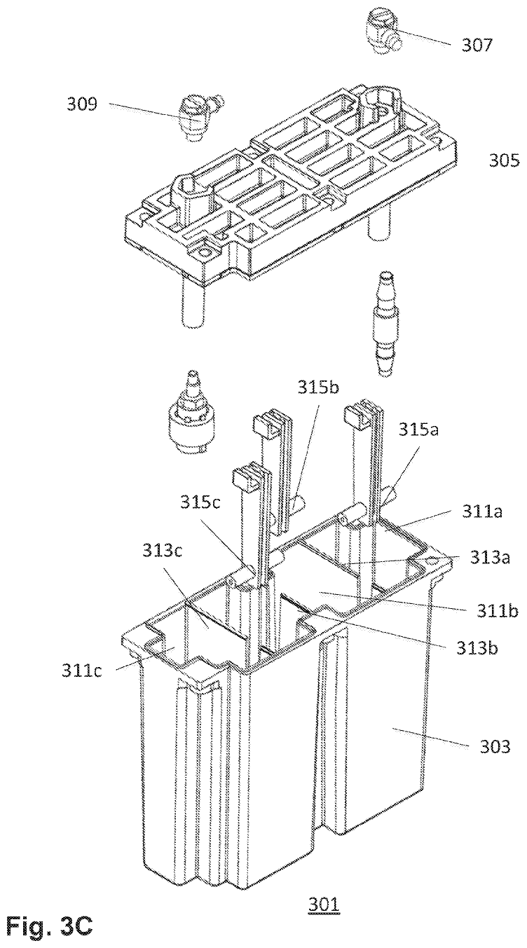

Provided in FIGS. 3A to 3C are various views of an example of a manifold comprising four expansion chambers in which the pressure flow pathways are indirect between the chambers, promoting the air flow to expand within all areas of the chamber. Manifold 301 comprises a base 303 and a lid 305 that are joined together to form a sealed internal compartment comprising the expansion chambers. Manifold 301 further comprises an inlet valve 307 for receiving the pressure generated by a pressure source (not shown). After the pressure passes through the internal chamber, it exits out an outlet valve 309 for distributing the pressure to the pressure regulator and further to the site where the pressure is needed (e.g., blood pressure cuff in blood pressure monitoring system).

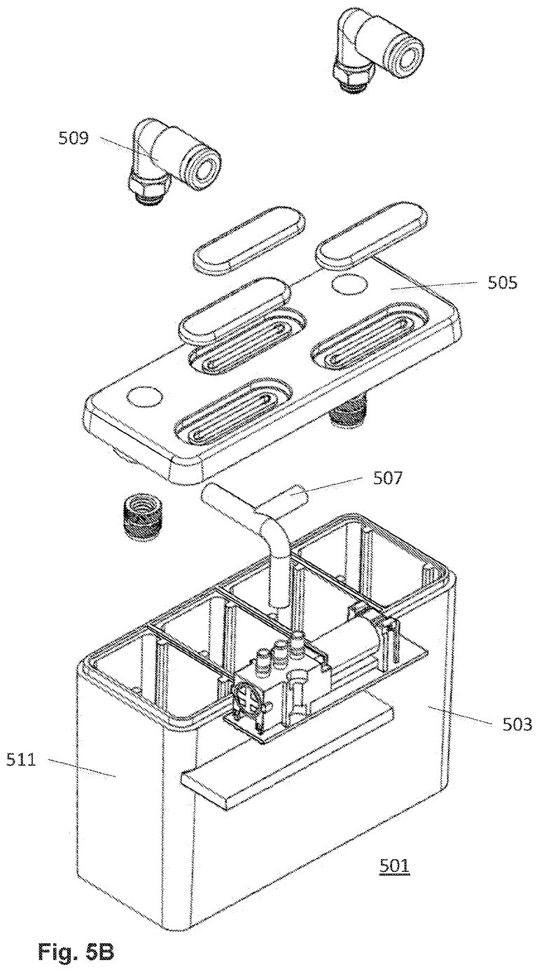

Within the internal compartment are four pressure chambers 311a-311d that are divided by three internal walls 313a-313c. Ports 315a-315c are provided that traverse through internal walls 313a-313c to connect the four chambers. Specifically, chamber 311a is connected to chamber 311b via port 315a; chamber 311b is connected to chamber 311c via port 315b; and chamber 311c is connected to chamber 311d via port 315c. The ports are configured such that there is not a straight path though the chambers. That is, port 315a is unaligned with port 315b; and port 315b is unaligned with port 315c. The configuration is set such that port 315b is askew towards a side port 315a; and port 315c is askew towards a side from port 315b. It should be understood that the ports can be askew in various directions, including towards a side; towards the lid; towards the bottom of the base; or any combination thereof. Further, ports can be provided on the top, bottom or side of an internal compartment to allow the pressure to bypass an inner wall that divides two expansion chambers. For example, see the exemplary manifold in FIGS. 5A and 5B, which has port connections within the lid of the manifold.

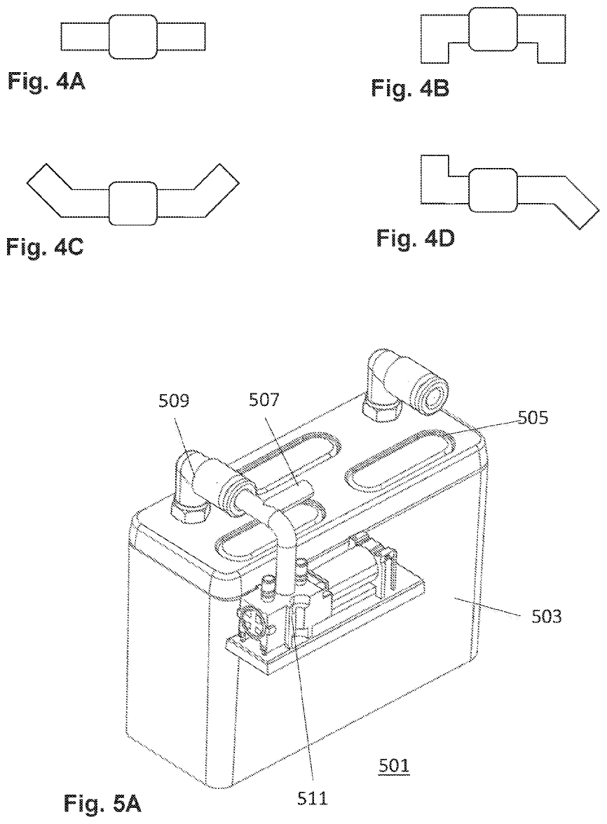

Provided in FIGS. 4A to 4D are examples of ports that can be utilized to connect one expansion chamber with another. The ports can be provided as a straight connector (FIG. 4A) or having a bend (FIGS. 4B, 4C, and 4D) such that the pressure transmits to another expansion chamber in an indirect line. The bend can have any angle between 1 and 180 degrees, such as a 90-degree angle (FIG. 4B) or 135-degree angle (FIG. 4C). Further, both ends of the port can have the same angle or a different angle (FIG. 4D). It should be further understood that the either end can be rotated about the longitudinal axis.

Having the ports between the expansion chambers askew and/or having bends within the ports can improve the ability of the expansion chamber to passively dampen the pressure pulsations. When the ports are aligned or near-aligned, some of the pressure will pass through the ports directly without expanding in the chamber, preventing the passive damping. When ports are askew and/or contain a bend, it prevents direct transfer of air flow from one port to another and promotes the air flow to expand into all areas of each expansion chamber. The more the air flow expands, the greater reduction in pulsation that can be achieved.

FIGS. 5A and 5B provide an example of a manifold in which the manifold inlet valve is in connection with both a release valve and the internal compartment. Manifold 501 comprises a base 503 and a lid 505 that are joined together to form a sealed internal compartment. Manifold 501 further comprises an inlet valve 507 for receiving the air pressure generated by a pressure source (not shown). Inlet valve 507 is connected to the internal compartment via an inlet port 509 and is also connect to a release valve 511. Release valve 511 can passively release volume of air or be controllable such that a controlled amount of volume released. When a passive release of pressure is utilized, the release valve port, the inlet valve port (and connectors between the ports) can be sized and configured to allow a relative volume of pressured air that enters into the chamber and a relative volume of pressured air that is released by the release valve.

In some implementations, like as shown in FIGS. 5A and 5B, it is beneficial to have the release valve before or concurrent with the internal chamber such that a greater volume of air that can be tolerated by the internal chamber can be generated by the pump, resulting in less prominent pressure pulsations to enter into the internal chamber. Generally, manifolds are built and configured to have an internal volume handle a volume of air that is appropriate for the task to be performed. For instance, a blood pressure monitoring system can be configured to handle low to high flow rates of pressured air.

When the release valve is before or concurrent with the internal chamber, the release valve can permit the use of a manifold with a manifold having an internal volume that is less than what is necessary to handle low to high flow rates air pressures. A more compact manifold may be desired in some situations, especially in portable medical devices (e.g., portable blood pressure monitoring system). Or alternatively, a traditionally sized manifold can be utilized and an excess volume of pressured air can be supplied to reduce the pulsation of pressure.

The release valve can release any amount of the volume provided by the pressure source. In various implementations, the release valve releases on or about 5% of the provided volume, on or about 10% of the provided volume, on or about 15% of the provided volume, on or about 20% of the provided volume, on or about 25% of the provided volume, on or about 30% of the provided volume, on or about 35% of the provided volume, on or about 40% of the provided volume, on or about 45% of the provided volume, on or about 50% of the provided volume, on or about 55% of the provided volume, on or about 60% of the provided volume, on or about 65% of the provided volume, on or about 70% of the provided volume, on or about 75% of the provided volume, on or about 80% of the provided volume, on or about 85% of the provided volume, on or about 90% of the provided volume, on or about 95% of the provided volume, or on or about 99% of the provided volume.

In various implementations, the release valve releases at least 5% of the provided volume, at least 10% of the provided volume, at least 15% of the provided volume, at least 20% of the provided volume, at least 25% of the provided volume, at least 30% of the provided volume, at least 35% of the provided volume, at least 40% of the provided volume, at least 45% of the provided volume, at least 50% of the provided volume, at least 55% of the provided volume, at least 60% of the provided volume, at least 65% of the provided volume, at least 70% of the provided volume, at least 75% of the provided volume, at least 80% of the provided volume, at least 85% of the provided volume, at least 90% of the provided volume, at least 95% of the volume, or at least 99% of the provided volume.

In some implementations, the volume of pressured air provided by the pump is in excess of the volume capacity of the system of expansion chambers. In various implementations, the volume of pressured air provided by the pump is at least 0.1× the volume capacity of the set of expansion chambers, the volume of pressured air provided by the pump is at least 0.1× greater than the volume capacity of the set of expansion chambers, the volume of pressured air provided by the pump is at least 0.25× greater than the volume capacity of the set of expansion chambers, the volume of pressured air provided by the pump is at least 0.5× greater than the volume capacity of the set of expansion chambers, the volume of pressured air provided by the pump is at least 0.75× greater than the volume capacity of the set of expansion chambers, the volume of pressured air provided by the pump is at least 1.0× greater than the volume capacity of the set of expansion chambers, the volume of pressured air provided by the pump is at least 1.25× greater than the volume capacity of the set of expansion chambers, the volume of pressured air provided by the pump is at least 1.5× greater than the volume capacity of the set of expansion chambers, the volume of pressured air provided by the pump is at least 1.75× greater than the volume capacity of the set of expansion chambers, the volume of pressured air provided by the pump is at least 2.0× greater than the volume capacity of the set of expansion chambers, the volume of pressured air provided by the pump is at least 3.0× greater than the volume capacity of the set of expansion chambers, the volume of pressured air provided by the pump is at least 4.0× greater than the volume capacity of the set of expansion chambers, or the volume of pressured air provided by the pump is at least 5.0× greater than the volume capacity of the set of expansion chambers.

In some instances, a release valve is utilized in the following method:
providing an excess of volume of pressured air
diverting a portion of the excess volume of pressured air into a release valve to be released prior to being transferred into a set of expansion chambers
transferring the remaining volume of pressurized air into the set of expansion chambers
transferring the remaining volume of pressurized air into a pressure regulator and/or into a medical device

What is claimed is:

1. A damping system for damping pressure pulsations from a pressure supply source, comprising:
a set of three or more expansion chambers that are connected by a set of two or more ports between each of the expansion chambers; wherein a first expansion chamber is configured to receive pressure and transfer that pressure to a second expansion chamber via a first port; wherein the second expansion chamber is configured to transfer that pressure to a third expansion chamber via a second port; wherein the first port and the second port are unaligned.

2. The active damping system of claim 1, wherein the second port is askew towards a side from the first port.

3. The active damping system of claim 1, wherein the second port is askew towards a top from the first port.

4. The active damping system of claim 1, wherein the second port is askew towards a bottom from the first port.

5. The damping system of claim 1, wherein the first port or the second port contains a bend.

6. The damping system of claim 1, wherein the set of three or more expansion chambers are provided within an internal compartment of a manifold.

7. The damping system of claim 6, wherein the manifold contains an inlet port in fluidic connection with a pressure source.

8. The damping system of claim 6, wherein the manifold contains an outlet port in fluidic connection with a pressure regulator.

8. The damping system of claim 6, wherein the manifold contains an outlet port in fluidic connection with a medical device.

10. The damping system of claim 9, wherein the medical device a pressure cuff for use within a blood pressure monitoring system.

11. A system for reducing pressure pulsations from a pressure supply source, comprising:
a manifold in fluidic connection with a pressurized air source, wherein the manifold is configured to passively dampen pressure pulsations via a set of one or more expansion chambers; and
a release valve provided after pressure source and either prior to or concurrently with an inlet to the manifold,
wherein the pressurized air source is configured to provide an excess volume of pressurized air that is greater than the volume capacity of the set of one or more expansion chambers, and wherein the release valve is configured to release the excess volume of pressurized air.

12. The system of claim 11, wherein the release valve is configured to control the volume of pressurized air to be released.

13. The system of claim 11, wherein the manifold contains an outlet port in fluidic connection with a pressure regulator.

14. The system of claim 11, wherein the manifold contains an outlet port in fluidic connection with a medical device.

15. The system of claim 14, wherein the medical device comprises a pressure cuff for use within a blood pressure monitoring system.

16. The system of claim 15, wherein the pressured air source is configured to provide an excess volume of pressured air that is at least 1.0× greater than the volume capacity of the set of expansion chambers.

17. A method of reducing pressure pulsations from a pressure supply source via the system of claim 11, the method comprising:
providing a volume of pressured air from the pressured air source, wherein the volume of pressured air includes an excess volume such that the volume of pressured air provided is greater than the volume capacity of the set of one or more expansion chambers; and
releasing the excess volume of pressurized air via the release valve and transferring the remaining volume of pressurized air into the set of one or more expansion chambers.

18. The method of claim 17, wherein the release valve releases at least 50% of the provided volume of pressured air.

19. The method of claim 17, further comprising further transferring the remaining volume of pressurized air into a pressure controller.

20. The method of claim 17, further comprising further transferring the remaining volume of pressurized air into a pressure cuff of a blood pressure monitoring system.
